(19) United States
(12) Patent Application Publication
(10) Pub. No.: US 2003/0229297 A1
(43) Pub. Date: Dec. 11, 2003

(54) Medical device for gripping an elongated member

(75) Inventors: Jes Christensen, Viby Sjaelland (DK); Per Hendriksen, Glumso (DK); Johnny Moller, Viby Sjaelland (DK)

Correspondence Address: COOK GROUP PATENT OFFICE, P.O. BOX 2269, BLOOMINGTON, IN, 47402

(73) Assignees: Cook Incorporated, Bloomington, IN; William Cook Europe APS, Bjaeverskov (DK)

(21) Appl. No.: 10/390,975

(22) Filed: Mar. 18, 2003

Related U.S. Application Data
(60) Provisional application No. 60/365,891, filed on Mar. 19, 2002.

(30) Foreign Application Priority Data
Mar. 18, 2002 (EP) 02076050.0

(51) Int. Cl. A61M 25/00

(52) U.S. Cl. 600/585; 604/164.01

(57) ABSTRACT

A medical device, for gripping an elongated member, comprises a plurality of fingers (16) having portions (28) between which the elongated member is to be gripped when the fingers are urged together into an engagement position. Each finger has a pair of side surfaces (22), each of which is to be urged towards a side surface of an adjacent finger when the fingers are urged into the engagement position. Adjacent side surfaces are shaped to prevent penetration of the space or slit (19) between two fingers by said elongated member. A single zigzag shape of the slit in the region of the head (11) of the vice, and a vice comprising four fingers and a single material are preferred.

### CROSS-REFERENCE TO RELATED APPLICATIONS

This application claims priority of provisional application Serial No. 60/365,891, filed Mar. 19, 2002.

### TECHNICAL FIELD

The present invention relates to the field of medical devices in general, and more particularly to methods and devices for the manipulation of elongated members, such as guidewires, during access to animal or human bodily passages such as blood vessels.

### BACKGROUND OF THE INVENTION

Medical guidewires have a small diameter, making it impractical for a physician to grip and manipulate such a guidewire directly by hand. This is a major disadvantage in an emergency where it is critical to the survival of patients that catheterisation of branched or convoluted blood vessels be an efficient procedure. The present invention concerns a vice, which is also known as a “torque” device, a “gripping” device, a “steering” device or “migration controller” device, and which is used to facilitate the manipulation of small diameter guidewires.

The gripping means described in the prior art are broadly divided into systems either using a rubber, or a mechanical mechanism such as the one disclosed in U.S. Pat. Nos. 5,851,189 and 5,392,778.

U.S. Pat. No. 5,851,189 to Forber discloses a torque device for attaching to and selectively gripping and releasing a guidewire to permit rotational and longitudinal manipulation of the guidewire and thereby to steer the guidewire through the vessels of a patient. The grip of the device over the guidewire is adjustable by means of two interconnected cylinders that can rotate with respect to each other. The first cylinder is the gripping device comprising a spindle with two fingers straddling the guidewire. The second cylinder is a cap that is rotated with respect to the first cylinder to force the distal end of the fingers to advance along a tapering bore in the cap and to close and grip the guidewire. The Forber device is simple and the guidewire is kept centred between the fingers of the spindle because the cap has a cylindrical bore through which the guidewire is threaded and maintained within the axis of the device. Furthermore, in the preferred embodiment of the Forber device, the spindle has only two fingers, so that the device can accommodate the full range of guidewire diameters with one single device. However, the Forber device has two main disadvantages. Firstly its gripping ability depends on the action of a spindle with only two fingers, and secondly, because the cap that maintains the guidewire centred within the spindle is an integral part of the gripping mechanism, manual access to the cap must be preserved at all times.

U.S. Pat. No. 5,392,778 to Horzewski discloses a similar device consisting of two cylinders that slide with respect to each other, one surrounding the distal part of the other. The inner cylinder comprises a multitude of prongs that encircle a guidewire that is threaded through a central passage of the inner cylinder. The outer cylinder, which comprises an inner ring encircling the prongs, can be advanced manually with respect to the first cylinder to close the prongs over the guidewire and provide a grip when the device is used to manipulate the guidewire. The Horzewski device has substantially the same disadvantages as the Forber device. The prongs are made of a resilient material, permitting the prongs to bend during compression by the outer cylinder, possibly compromising the gripping ability of the device. In addition, manual access to the outer cylinder that maintains the guidewire centred within the device has to be preserved.

One of the major disadvantages of the prior art devices is the possibility, during introduction of the guidewire, that it might get caught in the linear slits between the fingers or prongs of the device, causing the guidewire to become entrapped in the device, which could lead to kinking of the guidewire requiring it to be removed and replaced. Time might therefore be wasted, particularly in a situation where it is critical for the survival of a patient that a guidewire or other elongated member be introduced promptly in the vascular system.

### SUMMARY OF THE INVENTION

The present invention seeks to provide a device that can easily grip an elongated member such as a guidewire, that is self-centring, meaning that an elongated member could be maintained in the centre of the device without the help of any other component, that has an excellent ability to grip, that is capable of accommodating a wide range of elongated member's diameters, and that is yet simple and inexpensive to manufacture.

The foregoing problems are solved and a technical advance is achieved in a medical device, such as a vice for gripping an elongated member, the vice comprising a plurality of fingers encircling the elongated member when it is threaded through the central passage of the device. When the fingers are urged together into an engagement position, a portion of each finger grips the elongated member. Each finger comprises a pair of side surfaces, each of which is to be urged towards a side surface of an adjacent finger. Adjacent side surfaces are shaped with respect to each other, resulting in slits that are not straight and thereby preventing entrapment of the elongated member between the side surfaces of the fingers of the vice.

A vice formed in accordance with the present invention has a number of advantages. Since the side surfaces of adjacent fingers are shaped, and therefore the space or slit between the fingers is not straight, the elongated member is prevented to penetrate into the spaces or slits between the fingers, therefore avoiding one of the disadvantages of prior art devices where a guidewire could become entrapped between fingers or prongs having a straight space between them. A guidewire is, despite its high flexibility, to be regarded as straight on portions as short as the shaped part of the vice. The adjacent side surfaces of a pair of adjacent fingers could be shaped with all kinds of recesses and substantially complementary protrusions, thereby preventing penetration of the slits by a substantially straight elongated member. Kinking of the guidewire is thus totally avoided by the present invention. A further advantage of the vice is the self-centring ability: because the elongated member is unable to become entrapped in the slits between fingers, it is therefore kept in the central passage of the vice without the use of any other component of the vice. A device according to the present invention will accommodate a wide range of diameters and/or shapes of the elongated member, since even an elongated member having a diameter much smaller than the distance between the side surfaces of adjacent fingers would not be able to penetrate a slit that is not straight.

### BRIEF DESCRIPTION OF THE DRAWING

Embodiments of the present invention will now be described by way of example and with reference to the accompanying drawings in which:

FIG. 1 is a three-dimensional view illustrating the preferred embodiment of the vice in accordance with the present invention; and

FIG. 2 is a lateral view of the vice of FIG. 1 in accordance with the present invention; and

FIG. 3 is an end on view of the distal end of the vice of FIG. 1 in accordance with the present invention; and

FIG. 4 illustrates a longitudinal cross-sectional view of the vice at level A-A of FIG. 3, in accordance with the present invention; and

FIG. 5 is a three-dimensional view illustrating the inner side of a finger of the vice, in accordance with the present invention; and

FIG. 6 is a three-dimensional view illustrating a second embodiment of the vice in accordance with the present invention; and

FIG. 7 is a three-dimensional view illustrating a third embodiment of the vice in accordance with the present invention; and

FIG. 8 is a longitudinal cross-sectional view of a vice in accordance with the present invention illustrating features of adjacent side surfaces of adjacent fingers.

### DETAILED DESCRIPTION

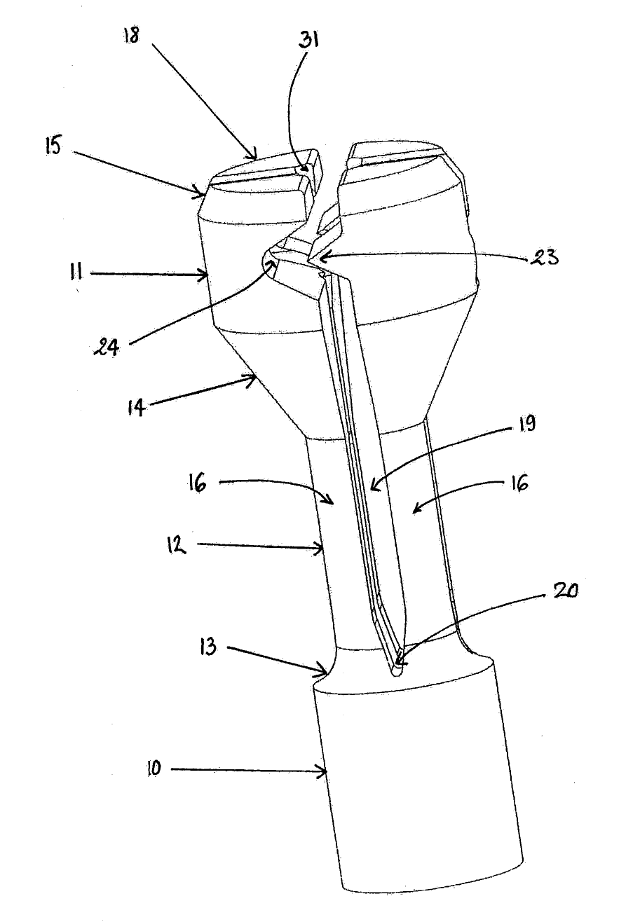

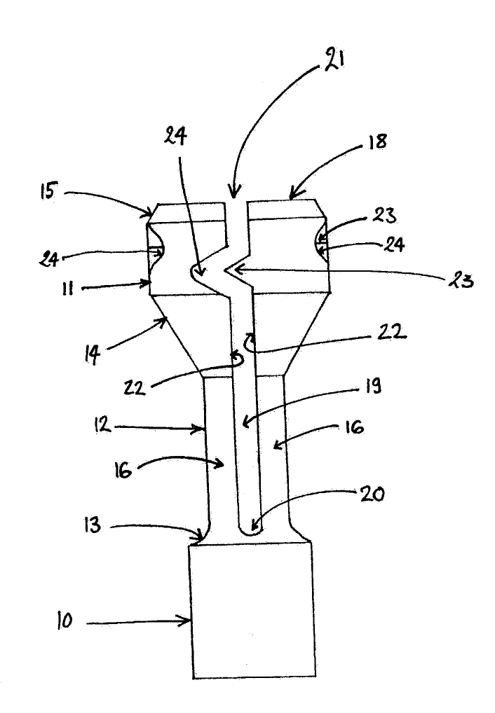

FIGS. 1 to 5 illustrate the preferred embodiment of the vice, which comprises three main parts: a base 10, a neck 12 and a head 11. Base 10 is preferably cylindrical and located in the proximal portion of the device. Neck 12 is preferably cylindrical and located between base 10 and head 11. Head 11, which is also preferably cylindrical, is located in the distal portion of the device. Proximal intermediate part 13 extends between neck 12 and base 10, and distal intermediate part 14 extends between neck 12 and head 11. The proximal intermediate part 13, which can have any shape, preferably tapers from the larger diameter base 10 to the smaller diameter neck 12. Alternatively, the diameter of neck 12 can be made equal to or larger than the diameter of head 11 and/or base 10. The distal intermediate part 14 has preferably a conical shape, but can have any other shape. Distal part 15 of the vice is preferably conical and has a diameter that decreases towards the distal end 18 of the device. Alternatively, distal part 15 can have any shape and be smaller than, equal to or larger than head 11.

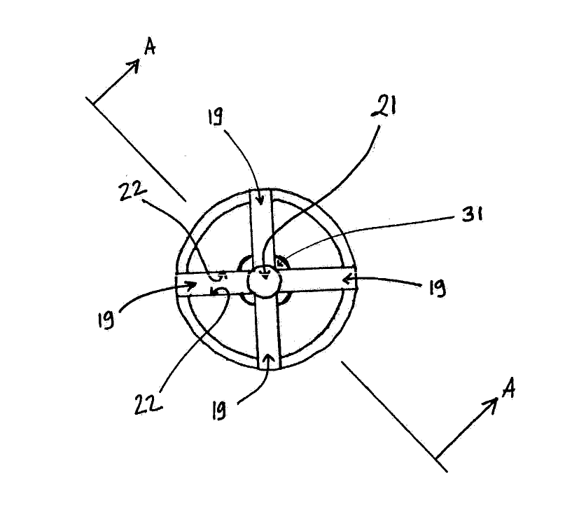

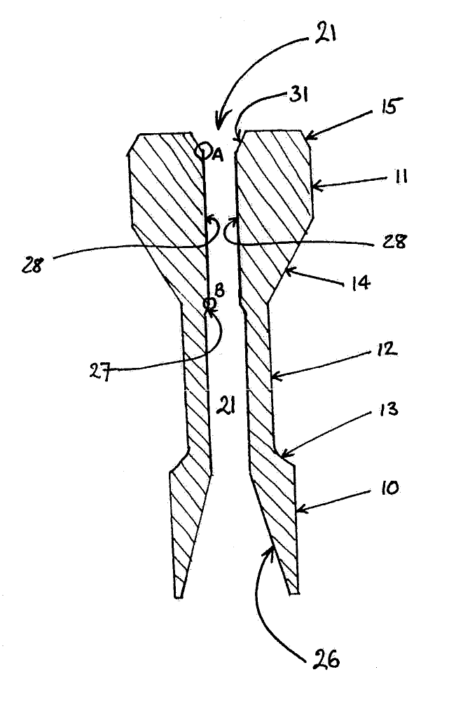

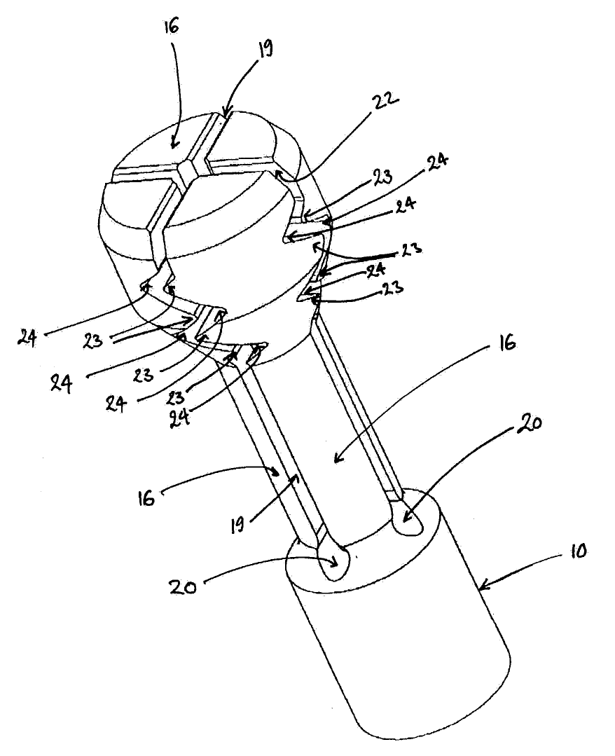

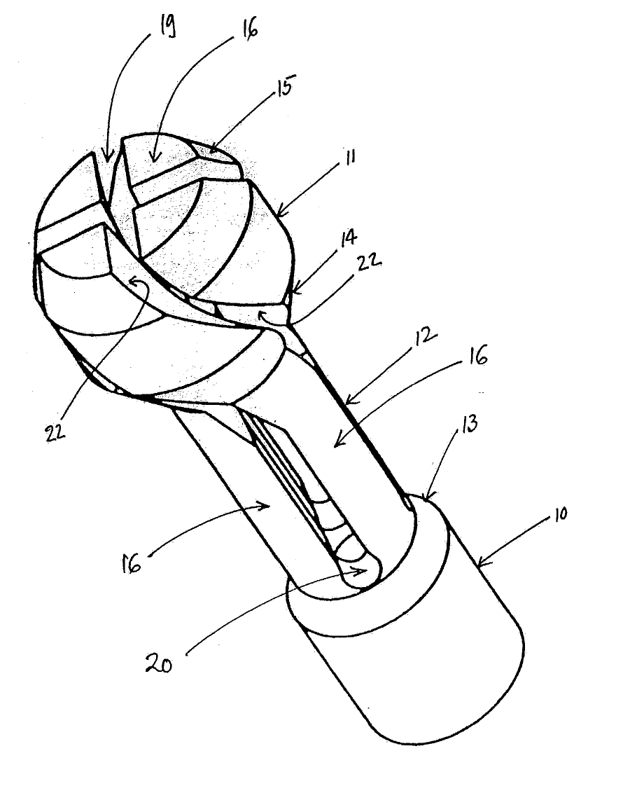

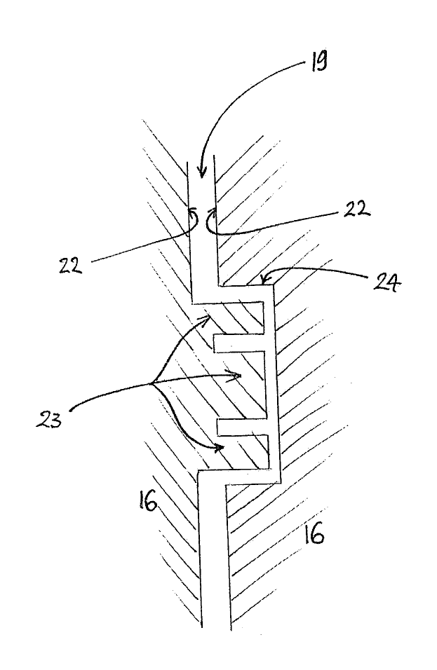

A vice is used for gripping elongated members such as a guidewire. The elongated member is threaded within a central passage 21 of the vice, as illustrated in FIG. 4. In FIGS. 3 to 5, the distal opening of the central passage 21 is illustrated as an enlarged distal conical bore 31, located within the distal part 15 of the vice, in order to facilitate the introduction of an elongated member through the distal opening of the central passage. It should be noted, however, that it is preferable to introduce the elongated member into the vice through the proximal conical bore 26. The part of the vice distal to base 10 comprises at least two fingers, preferably three fingers, and most preferably four fingers 16, each extending longitudinally from base 10 and each having a free distal end 18. It is to be noted that although a three-finger device provides a self-centring mechanism for the elongated member, the preferred embodiment of the present invention comprises four fingers because of the ease of manufacturing such a device. The gripping action of the device is achieved when the fingers 16 are urged together into an engagement position with the elongated member. The space between two adjacent side surfaces 22 of two adjacent fingers 16 delimits a slit 19 having a shape that is not straight, for preventing access of the elongated member therethrough. The number of slits between fingers 16 depends on the total number of fingers comprised in the device; for example, a vice with four fingers has four slits. The proximal part 20 of slit 19, located at the level of proximal intermediate part 13, has a shape that is for example triangular, as illustrated in FIG. 1, or rounded as illustrated in FIGS. 6 and 7. Proximal part 20 can also have any other shape. Because elongated members are prone to become entrapped between the fingers of a vice having straight slits, the vice of the present invention has slits that are not straight in shape. Features of the side surfaces 22, such as for example protuberance 23 and recess 24, are responsible for creating non-straight slits. The parts of the slit where the side surface features are located constitute the non-straight part of the slit. This non-straight part can extend over the entire length of the slit or preferably only over a fraction of its length. The non-straight part of the slit is preferably located in the distal portion of the vice, more preferably at the level of the distal intermediate part 14 and head 11, and most preferably at the level of the head 11 of the vice, as a single non-straight part. A single slit can also have multiple non-straight parts that are not contiguous. FIG. 6 is an example of a vice wherein the non-straight part of the slit is located in both the distal intermediate part 14 and the head 11. A vice formed with non-straight slits, in accordance with the present invention, makes it impossible for an elongated member to become entrapped between the fingers of the device. An exemplary feature of the side surface 22 consists of a protuberance 23 preferably located at the level of head 11 of one side surface 22 of a finger 16, which substantially complements a recess 24 on the adjacent side surface 22 of an adjacent finger 16, when the fingers are urged together in an engagement position. In the preferred embodiment, which comprises four fingers, the slits 19 that are diametrically opposed are identical. One of the main advantages of such an embodiment is that the manufacturing process is simplified and efficient, as it is possible, for example during electro-discharge machinery cutting, to cut in a single pass a pair of shaped and diametrically opposed slits. A second cut creates the other pair of shaped opposed slits, which have either an identical or a different shape than that of the former pair of slits. Therefore, the fingers of the vice of the preferred embodiment are all different. If opposing slits are not cut simultaneously, i.e. four cuts are then necessary to create four slits. In the latter case, two different pairs of identical fingers or four identical fingers are created. Alternative devices according to the present invention comprise fingers with identical or dissimilar side surfaces. For example, a finger can have a protuberance or a recess on both of its side surfaces. A protuberance or recess of one side surface may be offset longitudinally with respect to the protuberance or recess of the other side surface of the same finger. Further devices in accordance with the present invention comprise fingers having features on adjacent side surfaces of adjacent fingers that are not complementary. FIG. 8 illustrates one non-limitative example of non-complementary features. Producing a vice with complementary features of adjacent side surfaces has a major advantage with respect to ease of manufacture compared to production of a vice with non-complementary features, since in the former case, cutting of a slit results automatically in the appropriate shaping of the adjacent complementary features. This is not the case for side surfaces with non-complementary features, where multiple cuts are needed to create a slit.

FIG. 4 illustrates a longitudinal cross-sectional view of the vice of FIG. 3 at level A-A. Central passage 21 has three portions: a proximal portion that corresponds to base 10, an intermediate portion that corresponds to proximal intermediate part 13 and neck 12, and a distal portion that corresponds to distal intermediate part 14, head 11 and distal part 15. The proximal portion of the central passage 21 has a proximal bore 26, that is preferably conical, to facilitate introduction of an elongated member in a proximal to distal direction. The distal portion of passage 21 within the distal part 15 of the vice is preferably conical to allow for introduction of an elongated member in a distal to proximal direction. The proximal conical bore 26 has sloping edges, preferably at an angle of about 15° with respect to the longitudinal axis of the vice. Central passage 21, within neck 12 of the vice, is preferably cylindrical and has a diameter that is slightly larger than the diameter of the central passage 21 within head 11. Inner bore 27, at the junction of intermediate portion and distal portions of central passage 21, is preferably conical and has sloping edges preferably at an angle of about 15° with respect to the longitudinal axis of the vice. Distal inner bore 27 at the level of the distal part 15 of the vice is enlarged in a preferably conical shape with sloping edges preferably at an angle of about 30° with respect to the longitudinal axis of the vice. Passage 21 can be formed to have different shapes than the ones depicted above. For example and without limitations, passage 21 can have a transverse section that is square or elliptical in one or several portions of central passage 21. The edges of the side surfaces 22 at distal end 18 are optionally chamfered.

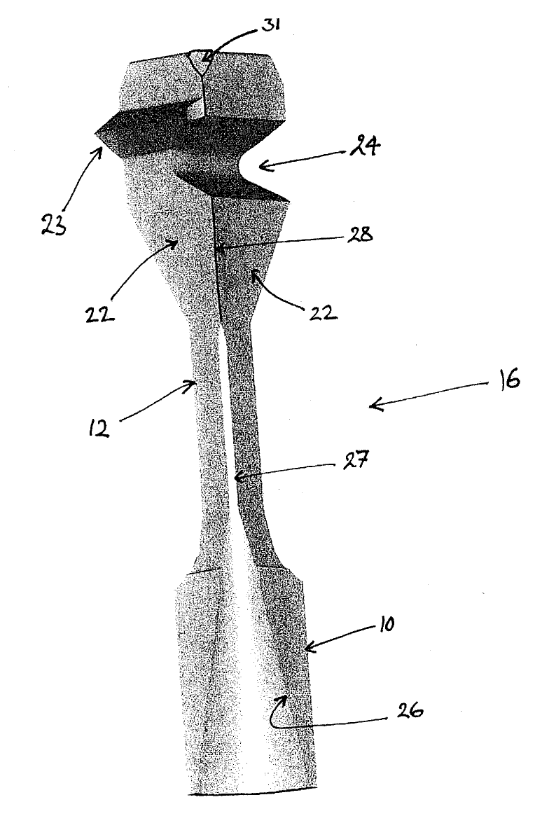

FIG. 5 illustrates the inner side of a segment of the vice comprising an exemplary finger 16 and part of the base 10 from which finger 16 extends. The cut out view of the inner side of the central passage 21 illustrates the proximal portion of central passage 21 having preferably a conical bore 26, continued distally by inner bore 27 of the intermediate portion of central passage 21 having preferably a cylindrical bore, in turn continued distally by the distal portion of central passage 21 which is delimited by the portion 28 of finger 16. The diameter of the distal portion of central passage 21, within the distal intermediate part 14 and head 11, is critical to determine the maximum diameter of the elongated member that can be used with such a device. Because of the gripping action that is caused by urging together the fingers of the vice into an engagement position, the area of maximum grip, in the case of an elongated member that has the same diameter as the critical distal portion of central passage 21, occurs between point A and point B as shown in FIG. 4. When the elongated member has a diameter inferior to the diameter of the distal portion of central passage 21, the maximum grip on the elongated member, when the fingers are in the engagement position, occurs at A. Depending on the amount of external force that is applied to the outer surface of the fingers at the engagement position, and depending on the material of which the fingers are made, the length of portion 28 that contacts the elongated member extends from point A to a point between A and B. Even for an elongated member having a diameter inferior to the diameter of the critical distal portion of central passage 21, the entire distance A to B contacts the elongated member when the external force is sufficiently high and/or the material of which the fingers are made is sufficiently flexible. Referring back to FIG. 5, the portion 28 is interrupted by recess 24. Alternatively, it is contemplated that protuberance 23 and complementary recess 24 have a transverse dimension that is less than the transverse dimension of the side surface 22 of finger 16 at that level, to allow for a continuous portion 28. It is also contemplated that portion 28, which is linear in the preferred embodiment, be shaped to improve gripping of the elongated member.

FIG. 6 depicts an alternative illustrative embodiment of the present invention wherein each finger 16 comprises several protuberances 23 and recesses 24 on one side surface 22 of a finger, to match complementary recesses 24 and protuberances 23 of an adjacent side surface, when the fingers 16 are urged together in an engagement position. The space so delimited between adjacent side surfaces has a double zigzag shape. It is to be understood that the shape of protuberances 23 and recesses 24 can vary to include, for example, hemispherical, spheroidal, cubical or other protuberances and respective complementary recesses. Moreover, although the protuberances and recesses are preferably located in the distal intermediate part 14 and head 11 of the vice, it is also within the scope of the present invention to form vices having protuberances and recesses which are located anywhere along the side surfaces 22 of each finger to form slits having either a single non-straight part or multiple non-straight parts that not contiguous.

In another embodiment of the present invention illustrated in FIG. 7, the fingers are shaped to create slits that have a helical shape in the distal part 15, head 11 and distal intermediate part 14 of the vice, thus creating a non-straight slit 19 which prevents elongated members to becoming entrapped between fingers 16 of the vice, when the fingers 16 are urged together in an engagement position.

The materials and methods of construction and the dimensions of the device are not essential and may vary. The material of which the vices are made is preferably, and not limited to, one of brass, stainless steel, copper, beryllium-copper alloy, or a polymer such as cellulose esters, polyesters, copolyesters, copolyester ethers (COPE) and polyethylene. Although it is preferable to use a single material to construct the vice in accordance of the present invention, it is within the scope of the present invention to construct vices either with a combination of materials, different parts of the vice being constructed from different materials, or with a mixture of at least two of the materials cited above. Furthermore, it is also to be understood that the vice of the present invention can be constructed for example from one single piece, or from several pieces constructed separately and subsequently joined, for example by pressing, bonding, welding or attaching the parts together in any other suitable way. Methods of manufacturing the metallic vices of the present invention include for example milling, electro-discharge machinery cutting, injection moulding or lathe turning. Alternatively, when for example injection moulding of a liquid crystal polymer is used, the device of the present invention is conveniently made from a number of pieces that are preferably identical, such as the piece illustrated in FIG. 5, and that are subsequently joined to form the vice of the present invention. The injection moulding method of manufacture, by decreasing dramatically the production costs of the device, is well suited for industrial production of the present device. The dimensions of the vice are not essential and are given below solely as examples applying to the illustrative embodiments of the present invention.

| | |
|---|---|
| Parameters | Approximate values in mm |

| | |
|---|---|
| Total length of the vice | 12.50 |
| Length of base 10 | 3.50 |
| Length of [base 10 + neck | 7.90 |
| 12] | |
| Diameter of base 10 | 3.20 |
| Depth of recess 24 | 0.90 |
| Length of distal part 15 | 0.53 |
| Width of slit 19 | 0.60 |

For convenience, the device of the present invention has only been described in the context of a medical use, but it should be kept in mind that the vice depicted above could also be used, for example, as a component of drill chucks or of mechanical pencils. For that reason, the dimensions, materials and methods of manufacture cited above are merely examples that should not be considered as limiting features of the present invention.

What is claimed is:

1. A medical device for gripping an elongated member, wherein the device comprises a plurality of fingers, wherein the fingers have portions between which the elongated member is to be gripped when the fingers are urged together into an engagement position, wherein each finger has a pair of side surfaces each of which is to be urged towards a side surface of an adjacent finger when the fingers are urged into the engagement position, and wherein adjacent side surfaces are shaped with respect to one another to prevent penetration therebetween by the said elongated member.

2. A device as claimed in claim 1, wherein adjacent side surfaces have shapes with respect to one another that are not complementary.

3. A device as claimed in claim 1, wherein adjacent side surfaces have shapes with respect to one another that are complementary.

4. A device as claimed in claim 1, wherein adjacent side surfaces of two fingers have shapes with respect to one another that are identical to the shapes of at least one other pair of adjacent side surfaces.

5. A device as claimed in claim 2, wherein adjacent side surfaces of two fingers have shapes with respect to one another that are identical to the shapes of at least one other pair of adjacent side surfaces.

6. A device as claimed in claim 3, wherein adjacent side surfaces of two fingers have shapes with respect to one another that are identical to the shapes of at least one other pair of adjacent side surfaces.

7. A device as claimed in claim 1, wherein the shapes of adjacent side surfaces with respect to one another are different in every pair of side surfaces.

8. A device as claimed in claim 2, wherein the shapes of adjacent side surfaces with respect to one another are different in every pair of side surfaces.

9. A device as claimed in claim 3, wherein the shapes of adjacent side surfaces with respect to one another are different in every pair of side surfaces.

10. A device as claimed in claim 1, wherein the space between adjacent side surfaces of at least one pair has a single zigzag shape.

11. A device as claimed in claim 3, wherein the space between adjacent side surfaces of at least one pair has a single zigzag shape.

12. A device as claimed in claim 4, wherein the space between adjacent side surfaces of at least one pair has a single zigzag shape.

13. A device as claimed in claim 7, wherein the space between adjacent side surfaces of at least one pair has a single zigzag shape.

14. A device as claimed in claim 1, wherein the space between adjacent side surfaces of at least one pair has a double zigzag shape.

15. A device as claimed in claim 3, wherein the space between adjacent side surfaces of at least one pair has a double zigzag shape.

16. A device as claimed in claim 4, wherein the space between adjacent side surfaces of at least one pair has a double zigzag shape.

17. A device as claimed in claim 7, wherein the space between adjacent side surfaces of at least one pair has a double zigzag shape.

18. A device as claimed in claim 1, wherein the space between adjacent side surfaces of at least one pair has a helical shape.

19. A device as claimed in claim 3, wherein the space between adjacent side surfaces of at least one pair has a helical shape.

20. A method of gripping an elongated member by clamping it within a device in which adjacent fingers are shaped to prevent penetration of the elongated member therebetween.
